(19) United States
(12) Patent Application Publication
(10) Pub. No.: US 2006/0127403 A1
(43) Pub. Date: Jun. 15, 2006

(54) Use of antibodies against tumor-associated antigen

(75) Inventors: Hans Loibner, Vienna (AT); Gottfried Himmler, Vienna (AT); Hellmut Samonigg, Graz (AT); Eugen Stermetz, Vienna (AT); Andreas Hauer, Vienna (AT); Gerda Redl, Rutzendorf (AT)

Correspondence Address: BIRCH STEWART KOLASCH & BIRCH, PO BOX 747, FALLS CHURCH, VA, 22040-0747, US

(73) Assignee: IGENEON KREBS-IMMUNTHERAPIE FORSCHUNGS-UND ENTWICK, Vienna (AT)

(21) Appl. No.: 10/524,520

(22) PCT Filed: Jul. 31, 2003
(86) PCT No.: PCT/AT03/00219

§ 371 Date: Oct. 31, 2005

(30) Foreign Application Priority Data
Aug. 12, 2002 (AT) A 1217/2002

(51) Int. Cl. A61K 39/395

(52) U.S. Cl. 424/155.1

(57) ABSTRACT

The invention relates to the use of a preparation based on an antibody directed against a tumor-associated antigen, for producing a medicament for the intra-operative treatment of tumor patients, in particular for the prophylactic treatment to prevent the dissemination of tumor cells. The invention further relates to a kit for the intra-operative treatment of tumor patients, comprising the medicament and means for diagnostically determining malignant tumor cells.

The invention relates to a new use of an antibody preparation as well as to a kit for the intra-operative treatment of tumor patients.

Tumors form due to the unchecked cell growth which leads to the formation of solid cell agglomerates in case of epithelial cells. In case of benign tumor tissue, it is assumed that the cell growth is limited and secondary tumors or metastases will not occur. In cancer diseases, however, malignant tumors form, and in the progressing stage secondary tumors and metastases occur. Most frequently, cancer forms with epithelial tumors occur which inter alia concern breast, stomach, intestines, pancreas, lungs, prostate and ovaries.

Cancer is a wide-spread disease and is lethal in many cases. The therapy of cancer usually comprises the removal of a solid tumor, and a further treatment which is to prevent and reduce, respectively, metastases. Besides surgery, the standard therapies include chemotherapy and radiation therapy. Despite the comprehensive therapy which often involves severe side effects, the success of treatment is insufficient. The relapse rate in intestinal cancer is approximately 45%. Metastatic epithelial cancer is considered to be nearly incurable. Therefore, in the treatment of cancer patients it is important to prevent, and reduce, respectively, the formation of metastases.

Tumor cells are capable of disseminating from primary tumors in body liquids and other organs. These disseminated tumor cells may be in their dormant state and often cannot be attacked by a chemotherapy (radiotherapy). Such a treated patient seems to be in a cured state, which is described as “minimal residual disease”. Dormant tumor cells, however, have a potential of forming metastases if they become growing and metastasizing cells.

Immunotherapy constitutes an innovative possible treatment of cancer patients. Both active and also passive immunotherapy are acknowledged measures for supporting the immune system.

The adaptive immune system of humans consists of two essential components, the humoral and the cellular immunity. The adaptive immune response partially is based on the clonal selection of B- and T-lymphocytes and in principle allows for the recognition of any desired antigen as well as for the build-up of an immunological memory. These characteristics of the adaptive immune system are generally usefully addressed in vaccinations.

Each B-cell produces an antibody with a defined binding specificity. This antibody is also present as a specific receptor in the membrane of the B-cell producing it. The humoral immune response against antigens recognized as foreign is based on the selective activation of those B-cells which produce such antibodies that can bind to an epitope of the respective antigen. For the antibody diversity, DNA rearrangements in the course of B-cell differentiation play a decisive role.

There are several possible ways of interfering in the immune system.

1. Passive Antibody Therapy:

For therapeutic purposes, it is possible to supply to an organism antibodies required for a certain function within this organism. This type of application is called passive immunotherapy, and it can be used in various medical indications, e.g. in the immunotherapy of cancer (Immunol. Today (2000), 21:403), intoxications (Toxicon (1998), 36:823; Therapie (1994), 49:41) and infections (Clin. Infect. Dis. (1995), 21:150). In these cases, antibodies can be used which either have been derived from appropriately immunized animals or can be recovered from cells by various biological or molecular-biological techniques (e.g. hybridoma technique, phage-display technique, etc.) via the immortalization of immunoglobulin genes.

2. Active Immunization:

To modulate the immune system, an immunization with antigens can be used. Antigens are molecules, molecule complexes or whole organisms to which antibodies can bind. Not all the antigens induce an immune response, i.e. not all the antigens are immunogenic. Certain small molecules are not registered by the immune system (haptens), such smaller molecules can be presented to the immune system in suitable form, and thus be made immunogenic. Such a method is the coupling of the hapten to an immunogenic molecule, a so-called carrier molecule. For an active immunization, also antibody preparations can be used, such as described in EP 1140168.

Tumor cells can be attacked by the immune system only to a limited extent, since they are hardly different from normal cells and specific antibodies therefore are missing. Much research is directed to the identification of suitable targets, i.e. target antigens, for the preparation of tumor-specific antibodies. The immunotherapy for the treatment of cancer then either comprises the passive therapy by the direct administration of the specific antibodies, or the active vaccination with suitable antigen-targets for stimulating the immune system and generating the specific antibodies in vivo.

One approach of relatively specifically destroying tumor cells is the passive immunotherapy with antibodies directed against tumor-associated antigens (TAA) (Immunology Today (2000), 21:403-410; Curr. Opin. Immunol. (1997), 9:717). Another approach of destroying tumor cells is an active vaccination which triggers an immune response against TAA. This immune response thus is also directed against the corresponding tumor cells (Ann. Med. (1999), 31:66; Immunobiol. (1999), 201:1).

Certain TAAs are defined as relevant “targets” for the development of immunotherapeutic agents for the prophylaxis and/or treatment of cancer. TAAs are structures which preferably are expressed on the cell membrane of tumor cells, thereby allow for a differentiation relative to non-malignant tissue, and thus can be viewed as targets for the diagnostic and therapeutic applications of specific antibodies.

In the course of the discovery and the subsequent characterization of various TAAs it has been found that they often have important functions for cancer cells. They allow the degenerate cells to have properties characteristic of the malignant phenotype, such as, e.g., an increased adhesion capacity, or an increased uptake of growth factors, which are highly important for establishing metastases. However, in certain stages, such antigens may very well also be expressed on normal cells where they are responsible for normal functions of these cells. An example of this is the Lewis Y carbohydrate antigen which appears on the plurality of tumors of epithelial origin, but also plays an important role during the fetal development of epithelial tissues. It has been shown that the expression of this antigen in lung cancer is associated with an unfavorable prognosis, since Lewis Y positive cancer cells apparently have a higher metastatic potential (N. Engl. J. Med. 327 (1992), 14).

In EP 0 528 767, the use of a humanized anti-Lewis Y antibody for the treatment of epithelial cancer has been described.

Among the further known tumor-associated carbohydrate structures, there are, e.g., all those Lewis antigens which are highly expressed in many types of epithelial cancers. Among them are Lewis x-, Lewis b- and Lewis y-structures, as well as sialylated Lewis x-structures. Other carbohydrate antigens are Globo H-structures, KH1, Tn antigen, TF antigen, the alpha-1,3-galactosyl epitope (Elektrophoresis (1999), 20:362; Curr. Pharmaceutical Design (2000), 6:485, Neoplasma (1996), 43:285).

Other TAAs are proteins which are particularly highly expressed by cancer cells, such as, e.g. CEA, TAG-72, MUC1, Folate Binding Protein A-33, CA125, EpCAM, HER-2/neu, PSA, MART, etc. (Sem. Cancer Biol. (1995), 6:321). Relevant TAAs often are surface antigens of epithelial cells which occur in larger numbers in growing cells, such as fetal tissue, and also in tumor tissue.

Direct therapeutic applications of antibodies against TAA are based on passive immunotherapies, i.e., a specific antibody is systemically administered in a suitable amount to cancer patients, and has an immunotherapeutic effect. The biological half-life of such agents will depend on their structure and is limited. Therefore, it is necessary to carry out repeated applications. When using xenogenic antibodies (e.g. murine monoclonal antibodies, MABs) however, this can lead to undesired immune reactions which may neutralize a possible therapeutic effect and may cause dangerous side effects (anaphylactic reactions). Therefore, such immunotherapeutic agents can be administered for a limited time only.

A better tolerance is obtained by reducing the xenogenic structures of the antibody and by introducing human structures, e.g. with chimeric or humanized antibodies. Also systems for producing specific human antibodies are being developed. Thus, according to the prior art, certain cell lines, organisms or transgenic animals can produce human antibodies.

Different publications deal with the risk of metastasis formation by the liberation of tumor cells as it may occur with a surgical treatment. As described in Clin. Cancer Res. 4(2), 343-8 (1998), particularly during a resection of a tumor, tumor cells can be determined in a blood sample. A direct link is seen between the intra-operative dissemination of hematogenic tumor cells and the “minimal residual disease” as well as the formation of metastases. In Semin. Surg. Oncol. 20(4), 329-33 (2001) it has therefore been suggested to take additional perioperative measures of antibody therapy or cytotoxic therapy.

A combined treatment of tumor patients has been described in WO 00/69460. Prior to a surgical treatment, patients at first are treated for 8-24 weeks with an antibody preparation combined with a cytotoxic agent, at weekly intervals, so as to reduce the tumor size. After the tumor resection has been effected, the patient again is subjected to the combined immunotherapeutic treatment. This perioperative treatment at first comprises a complete treatment cycle weeks before surgery, and later on, a further cycle weeks after the surgery. In this document, antibodies are administered which lead to the cell death via an ADCC or a CDC function (cf. page 5, lines 10-17). The thus treated patients exhibit an improved survival rate as well as a reduced spreading of the tumor (TTP).

According to US 20010006618, antibody preparations have been suggested for diagnostic use during a surgical, intravascular, laparoscopic or endoscopic intervention. Intra-operative lesions are to be detected by using a labelled antibody preparation. In doing so, the labelled antibody is administered within 48 hours prior to the intervention, and after the surgery, the patient is examined with a view to the possible dissemination of the labelled antibody. In addition to its use for detecting the tumor, the labelling substance may also be used for treating the tumor, e.g. by using a photoactive reagent as labelling substance which after binding the antibody, can be activated by the action of light.

According to U.S. Pat. No. 4,643,971, a selection of tumor-specific antibodies has been suggested for determining biliary cancer prior to a respective surgery.

WO 01/23005 relates to the administration of antibodies which are conjugated with a dye so as to illustrated the tumor rim. The antibody dye conjugates preferably become enriched in the rim region of the cell tissue of a tumor and thus allow for an optical imaging of the rim region (cf. p. 8, 1^{st} complete paragraph).

The invention has as its object to improve the treatment of cancer patients in so far as the dissemination of tumor cells and the formation of metastases, respectively, are largely suppressed.

According to the invention, this object is achieved by the subject matter of the claims.

The use according to the invention relates to an antibody-based preparation for the production of a medicament, which antibody is directed against a TAA. This medicament is administered within the scope of surgical interventions for the intra-operative treatment of tumor patients, wherein tumor cells are immunocomplexed. The immunocomplex formation occurs during surgery, i.e. primarily on tumor tissue or cells which become intra-operatively accessible.

The intra-operative treatment is the prerequisite for the functional activation of the immune system against the tumor cell, whereupon the latter is immediately lysed. The dissemination of tumor cells which may occur during certain surgical procedures is then largely prevented. The fact that a dissemination of tumor cells may even occur during the biopsy, i.e. during a relatively slight invasive surgical intervention, has, e.g., been shown in female breast cancer patients. There, proof of disseminated tumor cells was not possible before surgery, after the biopsy, the level of circulating epithelial cells which correspond to disseminated tumor cells, increased highly in some instances.

According to the invention, the use of the medicament is primarily a prophylactic one so as to prevent the dissemination of tumor cells in secondary organs or in bone marrow.

The treatment according to the invention comprises both prophylactic and also therapeutic measures. The treatment does not only serve human medicine, it may also be indicated in surgical interventions on various mammals.

The term “intra-operative treatment” according to the invention is intended to mean the treatment of tumor patients which ensures a high antibody titer for the duration of a surgery. Practically, the antibody preparation is administered only a few hours before, i.e. immediately while the patient is being prepared for the surgery, or during the surgery itself. The treatment usually is carried out within 24 hours prior to surgery, preferably within 8 hours, more preferred within 4 hours, most preferred within one hour before the surgical intervention. The intra-operative treatment according to the invention thus differs from a perioperative treatment of the prior art which provides for a repeated treatment of the patient over several weeks prior to surgery, as well as a further treatment period after surgery.

According to the invention, surgical interventions are provided for the partial or complete tumor resection of solid tumors. Tumor tissue preferably is removed with a no-touch surgical technique so as to reduce the risk of the dissemination of tumor cells into the periphery right from the beginning.

Immediately before and during the surgical intervention, according to the invention the tumor patient usually will receive a single dose of an antibody preparation so as to achieve a certain serum content. Starting from a specific immunoglobulin content of approximately 10, preferably at least 50, most preferred more than 100 μg/ml serum, a protection against a possible spreading of tumor cells into the circulatory system is thus built up. After an intravenous treatment, the antibody titer is practically immediately available. Further systemic treatments comprise the intramuscular, subcutaneous, intradermal or mucosal, such as oral or nasal administration. In doing so, a delayed availability must be counted on. The antibody preparation can also be locally administered, i.e. to the tumor tissue and/or in the wound area after removal of the tumor. Various modes of administration may appropriately be combined, e.g. an i.v. infusion shortly before surgery, as well as a local dose directly applied into the wound region. Practical application means for the localized administration comprise, e.g., ready-to-use applicating means, such as catheters, syringes or sprays, suitable for distributing liquid medicaments.

Particularly in endoscopic or microinvasive surgical methods, the local application is advantageous. Thus, during biopsy, the medicament preferably is introduced via the puncture channel, or in parallel, via appropriate syringes or catheters, respectively.

Small tumors or a sample of a tumor tissue are usually removed by biopsy. On the basis of the biopsy material, the pathologist will diagnose whether the tissue is from a benign or a malignant tumor. Patients who are subjected to a biopsy as such are not yet subjected to a cancer therapy. According to the prior art, the patient will receive respective medicaments only after a cancer disease has been positively diagnosed. According to the invention, however, a high-risk patient will already receive the appropriate prophylactic treatment so as to exclude a dissemination of tumor cells due to the biopsy itself.

The medicament used according to the invention as such is well tolerable and usually is administered to the tumor patient only once. The prophylaxis therefore is also indicated if the risk of a cancer disease has not yet been confirmed, and if the surgical intervention will be carried out to decide on the malignancy of a tumor. Besides the usual diagnostics by the pathological finding, according to the invention there exists the further possibility to determine the immune complexes formed and to obtain in this manner an indicator for the malignancy of the tumor. A prerequisite for this is that the antibody used is directed against a TAA which distinguishes benign from malignant tumor tissue.

In case of a cancer disease, according to the invention immune complexes with tumor tissue, or with tumor cells, respectively, are obtained which are determinable not only at the tumor tissue, following resection of the tumor, but also in peripheral body fluids. After the treatment, blood or serum samples of the patient can be qualitatively and/or quantitatively assayed for immune complexes. The analysis methods are per se common analysis methods with fractionation and enrichment of the immune complexes and/or immunoreaction with a specific antibody, such as one directed against the Fc portion of the antibody employed for the treatment. If the antibody used comprises murine structures, the former can also be bound with an anti-murine antibody, as defined by a capture step. The detection of the binding usually is effected with an appropriate antibody labelling, such as a fluorescence label, or with a color reaction.

According to the invention, furthermore, a kit for intra-operative treatment of tumor patients is provided, which kit comprises
a) a medicament based on an antibody, directed against a tumor-associated antigen, and
b) a means for the diagnostic determination of malignant tumor cells which are immunocomplexed with the antibody.
   The antibody of the kit preferably is a native, i.e. a functionally active, antibody. This antibody thus preferably has not an adhered label or other detection agent so as not to impair its functionality.

The inventive use preferably is effected with antibodies directed against an epitope of a surface antigen of a tumor cell. Optionally, a TAA from cells of solid tumors, or a panel of TAAs is chosen, in particular individual TAAs of the tumor of the respective patient. Patients having primary tumors can be treated just as well as patients with secondary tumors.

As a rule it must be assumed that by an antigen which imitates a proteinaceous epitope of a TAA, a polypeptide of at least five amino acids is to be understood.

Among the epitopes of the inventively used antibody, preferably there is at least one epitope of a human antigen, selected from the group of the peptides or proteins, in particular EpCAM, NCAM, CEA and T cell peptides which preferably are derived from tumor-associated antigens, furthermore, the carbohydrates, in particular Lewis Y, Sialyl-Tn, Globo H, and the glycolipids, in particular GD2, GD3 and GM2. Preferred epitopes are derived from antigens which are specific for epithelial tumors and, e.g., occur increasedly in breast cancer, cancer of the stomach and intestines, of the prostate, pancreas, the ovaries and the lungs. Among the preferred epitopes are those which primarily trigger a humoral immune response, i.e. a specific antibody formation in vivo. Preferably, the immunogenic antibody according to the invention can also trigger a T-cell specific immune response, whereby, as a reaction to the administration of the antibody, antibodies not only of the IgM class, e.g., are formed, but also of those of the IgG-class.

As an alternative, especially those antigens can be selected as epitopes as defined by the invention, which generate a T cell specific immune response. Among them are primarily also intra-cellular structures or T cell peptides, respectively. The intra-operative treatment according to the invention may, e.g., also be used to reduce the volume of a pleural effusion, i.e. an accumulation of fluid in the pleural cavity. Pleural effusions often occur in patients who suffer from epithelial cancer.

Further preferred proteinaceous epitopes which are particularly expressed on cancer cells of solid tumors are, e.g., TAG-72, MUC1, Folate Binding Protein A-33, CA125, HER-2/neu, EGF receptors, PSA, MART, etc. (cf. e.g., Sem. Cancer Biol. 6 (1995), 321). Moreover, also so-called T cell epitope peptides (Cancer Metastasis Rev. 18 (1999), 143; Curr. Opin. Biotechnol. 8 (1997), 442; Curr. Opin. Immunol. 8 (1996), 651) can be used.

Suitable epitopes are expressed at least in 20%, preferably at least in 30% of the cases of tumor cells of a certain cancer type, further preferred in at least 40%, in particular in at least 50% of the patients.

Carbohydrate epitopes preferred according to the invention are tumor-associated carbohydrate structures, such as the Lewis antigens, e.g. Lewis x-, Lewis b- and Lewis y-structures, as well as sialylated Lewis x-structures. Furthermore, also Globo H-structures, KH1, Tn antigen, TF antigen, the alpha-1,3-galactosyl epitope are preferred carbohydrate antigen structures within the scope of the present invention.

A particularly good target for the inventively used antibody is the Lewis Y antigen. Particularly preferred is a humanized antibody, such as described in EP 0 528 767. This antibody recognizes Lewis Y antigen on tumor cells, or on surface receptors of tumor cells, respectively.

It has been found that the surface receptors of a tumor cell with an aberrant glycosylation form relevant epitopes as defined by the invention whereby this glycosylation can be functionally blocked by antibodies. This does not only apply to one certain surface receptor, such as the EGF receptor or Her-2/neu receptor. Practically all the tumor-specific receptors which are characterised by the aberrant glycosylation are simultaneously blocked. Among them are, e.g., all the receptors of the EGF receptor family, the CD55 (791Tgp72/DAF-decay accelerating factor) receptor, the transferrin receptor and the P-glycoprotein. Thus, the tumor cell is attacked on the basis of different mechanisms of action.

It has also been found that antibodies directed against an aberrant glycosylation bind to several receptors of the family of the EGF receptors in a functional manner and that thus the signal cascade for the induction of cell growth can effectively be blocked. In Austrian application A 995/2002 it could be shown that in particular the erk1 and erk2 isoforms of the MAP kinase can be functionally bound by the antibodies employed according to the invention. The binding of the growth factors to the receptors was thus prevented or reduced, respectively. Compared to the immunotherapy with antibodies against the proteinaceous extracellular part of the EGF receptor, this treatment is more specific since the unusual tumor-associated carbohydrate structures are missing on EGF receptors of normal cells. On the other hand, the treatment is more universal, since simultaneously different receptors with the same aberrant glycosylation are blocked.

By this use according to the invention, thus also the mitogenic stimulation of a cancer cell by EGF or heregulin is prevented. The specific binding of the antibody to a tumor-associated glycosylation of cancer cells blocks the interaction of the receptors of growth factors with their physiological ligands and inhibits the signal transduction through these receptors, and thus, the cell growth.

Simultaneously, such an antibody is capable of specifically attacking the tumor cell on account of its action within the humoral and cellular immune system. Tumor cells which express the EGF receptor, or receptors of the EGF receptor family, respectively, according to the invention are specifically bound and can be lysed.

Methods for locating suitable antigenic structures, modelling and preparation of the TAA-derived peptides, polypeptides or proteins, or of nucleic acids coding therefore, respectively, furthermore lipoproteins, glycolipids, carbohydrates or lipids are known to the person skilled in the art and can be provided for the respective tumor-specific structure without undue experimental expenditures. Furthermore, the methods of producing the specific antibodies are known which are suitable according to the invention.

In a special embodiment, an antibody mixture of various antibodies having specificity for TAA, in particular for at least two equal or different epitopes of an adhesion protein, such as a homophilic cellular membrane protein, such as EpCAM, is used. Likewise, a combination of antibodies having specificity for at least one TAA epitope of the Lewis carbohydrate antigens, in particular Lewis Y, is preferred.

According to the invention, the relevant antibody is primarily employed for passive immunization, and in particular is administered only once. Thus, no special side effects are expected, even if the inventive antibody is derived from a non-human species, such as a murine antibody. However, it is expected that a recombinant, chimeric, such as a humanized antibody combined with murine and human components, or a human antibody will be particularly tolerable for the administration on humans.

By the term “antibody”, antibodies of any type are to be understood, in particular monospecific or polyspecific monoclonal antibodies, or also chemically, biochemically or molecular-biologically produced antibodies, or polyclonal antibodies having a certain specificity, such as an immune serum or a fraction of an immune serum.

The antibody used according to the invention usually is a native antibody which possibly has been isolated from an organism or patient. Native antibodies are hetero-tetrameric glycoproteins assembled of two identical light chains and two identical heavy chains.

Yet, also an antibody derivative may be used which preferably is selected from the group of antibody fragments, conjugates, homologues or derivatives, yet also complexes with additional effector functions. In any event it is preferred for the antibody derivative to contain at least parts of the Fab fragment, preferably together with at least parts of the F(ab′)₂ fragment, and/or parts of the hinge region and/or of the Fc portion of a lambda or kappa antibody.

Furthermore, also a single-chain antibody derivative, such as a so-called single chain antibody having effector functions as defined by the invention may be employed. The antibody according to the invention preferably is of the type of an immunoglobulin, such as an IgG, IgM, IgA or IgD.

According to the invention, the antibody binds directly to a tumor cell or to metastases or micrometastases. An thus formed immune complex of the antibody is the prerequisite for the humoral and cellular activities of the immune system, expressed by an antibody-dependent cellular cytotoxicity (ADCC) and/or a complement-dependent cytotoxicity (CDC) effector function. These effector functions are determined by means of standard tests.

High-affinity antibodies are preferred according to the invention. In particular, antibodies are used which bind with an affinity corresponding to a dissociation constant of below a Kd value of 10⁻⁶ mol/l, preferably less than 10⁻⁷ mol/l, most preferred 10⁻⁸ mol/l, or less.

A possible treatment objective is the effective binding and reduction of tumor cells so as to prevent their dissemination as far as possible. Simultaneously, also particularly the disseminated tumor cells are attacked. The number of tumor cells, or micrometastases, respectively, detectable in blood, bone marrow or organs is significantly reduced by prophylaxis and therapy according to the invention. The formation of metastases is to be retarded thereby, and their growth is to be at least slowed down. Thus, by the immunotherapy according to the invention, the relapse-free life span and, thus, also the total survival time of the patients can be increased. An indicator for the success of the treatment is the significant reduction of tumor cells in blood, serum or bone marrow.

The medicament used according to the invention advantageously is provided in a suitable formulation. Preferred are such formulations with a pharmaceutically acceptable carrier. The latter comprises, e.g., auxiliary substances, buffers, salts and preservatives. Preferably, a ready-to-use infusion solution is provided.

Since an antibody is comparatively stable, medicaments based on antibodies or their derivatives have the substantial advantage that they can be put on the market as storage-stable solutions or as a formulation in a ready-to-use form. The latter is preferably storage-stable at refrigerating temperature up to room temperature. The medicament used according to the invention may, however, also be provided in frozen or lyophilized form which may be thawed or reconstituted, respectively, upon demand.

The antibody solution provided may be administered intravenously as a bolus injection or also in diluted form. The medicament may, e.g., be prepared as an infusion preparation in a 1:10 to 1:100-fold dilution with physiological saline solution.

To saturate the relevant surface antigens of the tumor cells, usually a high dose of at least 50 mg, preferably at least 100 mg, most preferred at least 200 mg is administered per patient. The maximum dose is limited by the tolerability of the antibody and will depend on its specificity and avidity. Humanized antibodies and human antibodies, respectively, are the best tolerable. A dose of up to 1 g or in some cases of up to 2 g per patient and treatment may very well be advantageous.

Usually the patient will be further examined after the surgical intervention so as to possibly carry out a suitable cancer treatment. Should it turn out after the surgical intervention that the tumor has the relevant TAA, an immediate further treatment with the same antibodies that had been used intra-operatively and/or with immunotherapeutically active other antibodies may be carried out.

The usual treatment for passive immunotherapy comprises repeated infusions at regular intervals, such as weekly for a period of time of from 6 to 24 weeks, at a dose ranging from 1 to 10 mg/kg, preferably ranging from 2 to 6 mg/kg. The treatment is preferably repeated at certain time intervals, corresponding to the half-life of the antibody used which usually is in the range of from 5 to 30 days. By a special derivatization of the antibody it is possible to lengthen the half-life to up to several months, and to thereby lengthen the treatment intervals accordingly.

The concentration of the active substance of the medicament will depend on its tolerability. A particularly well tolerated preparation based on a humanized antibody can be administered directly to the patient in a high concentration and without being further diluted. By the preferred concentration in the range of from 0.1% to 10%, preferably 1% to 5%, it is possible to keep low the administered volume and the respective infusion time.

The combination with known adjuvant treatment methods is quite common. Among them are agents for radiotherapy or chemotherapy, such as the monotherapy or polytherapy. For reasons of the different mechanisms of action, the immunotherapy preferably is combined with the polychemotherapy.

Agents preferably used for chemotherapy are alkylating pharmaceutical preparations. Thus, e.g., agents containing taxane, anthracyclines or platinum are preferred. All the conventional preparations which are employed for the various cancer treatments can be further combined according to the invention. The chemotherapeutic agents usually are administered intravenously or perorally. Peroral administration forms of the chemotherapeutic agents possibly can also be administered with a peroral form for the immunotherapy according to the invention as a combination preparation.

In the following, the preparation of a medicament produced according to the invention as well as its intra-operative use are described by way of example. The following examples and figures shall further explain the present invention, but not restrict it.

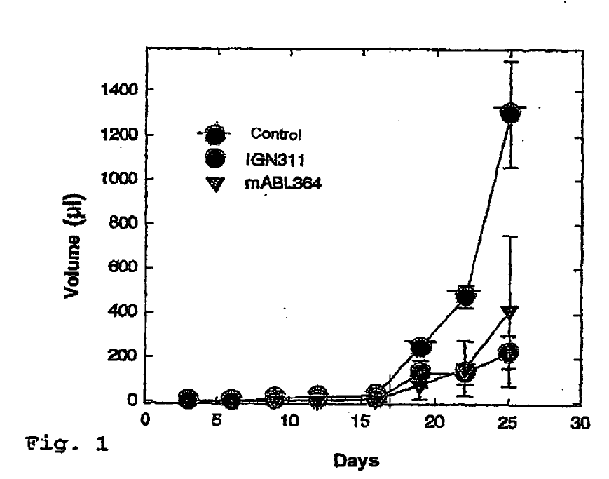

FIG. 1 shows the inhibition of xenograft growth by IGN311 and ABL364 after inoculation with 1×10⁶ A431 cells in mice.

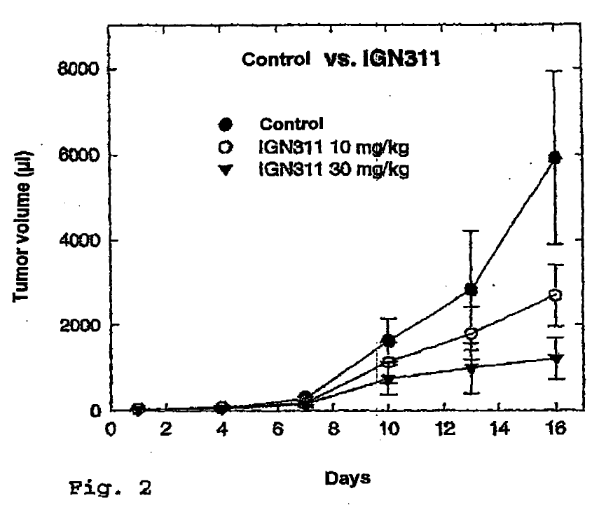

FIG. 2 shows the inhibition of xenograft growth by IGN311 after inoculation with 3×10⁶ A431 cells in mice. The treatment with the antibodies was carried out within one day after dissemination of the tumor cells.

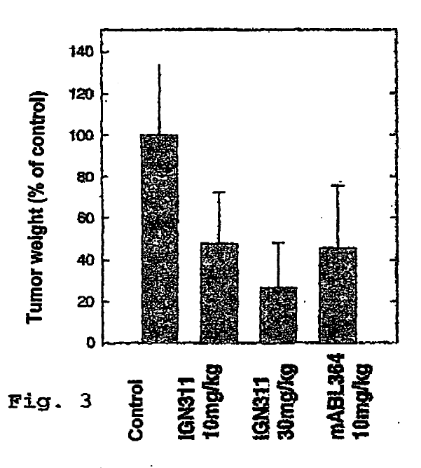

FIG. 3 shows the tumor weigths without and with a treatment with IGN311 and ABL364.

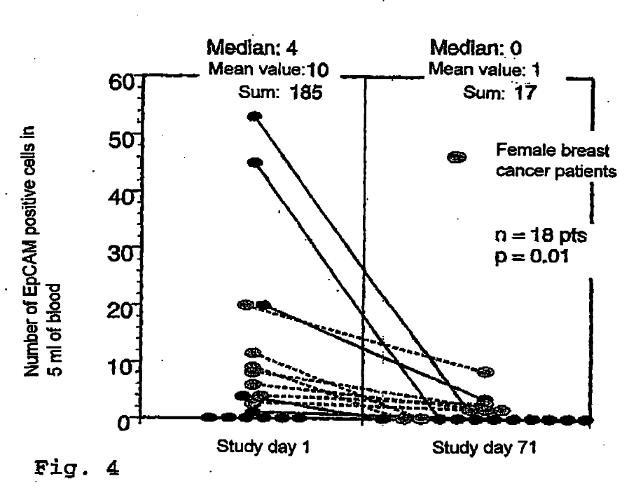

FIG. 4 shows the reduction of the circulating EpCAM positive cells in blood after treatment with a murine IgG2a Mab with Ep-CAM mimicking properties.

### EXAMPLES

### Example 1

### Preparation of a Lewis Y Antibody IGN311

IGN311 is a humanized antibody derived from Br55-2 murine IgG3 antibody. This murine antibody is derived from the hybridoma cell line BR55-2 (BR55-2/IgG3), deposited on Feb. 17, 1987, at the American Type Culture Collection, Rockville Md. 20852, USA, under ATCC HB 9324.

The humanized antibody was prepared by CDR grafting (Cancer Research 56, 118-125, Mar. 1, 1996). As constant region of the heavy chain, human IgG1 was employed. The humanization was effected according to EP 0 528 767. The antibody was produced in SP2/0 cells.

The IGN311 obtained proved to be highly active: Human complement was activated with a CDC approximately 10-fold increased relative to the murine antibody so as to lyse Lewis Y-positive tumor cells. The activity of the IGN311 was tested against a selection of Lewis Y-positive human tumor cell lines in the presence of human complement (CDC) (SKBR5, breast cancer, SW948, colon cancer; SW2, small cell lung cancer; and MCF7, breast cancer). The ADCC was also tested against a certain selection of tumor cell lines. The binding affinity remained approximately equal also after humanization.

The product was formulated in phosphate-buffered saline solution and filled into containers at a concentration of 10 mg/ml. Depending on its respective use, this stock solution will be diluted before being used. For the i.v. product, the solution is diluted 1:50 with physiological saline solution. If the preparation is to be applied locally in the wound area, the preparation will be used without being diluted.

Application During Tumor Resection

Patients diagnosed with a cancer of the intestines are prepared for the surgical removal of the tumor. Before the treatment, a blood sample is taken. Treatment with the i.v. product is effected immediately before surgery over a period of time of 2 hours. The dose administered was 50 mg. Subsequently, the tumor is removed within 4 hours.

Blood samples are taken immediately after the induction of anaesthesia, after tumor resection and 24 hours after surgery. The tumor cells are enriched from the fresh blood samples, applied to slides and stored at −20° C. until immunocytochemical analysis.

A sample of the removed tumor tissue is prepared for the immunohistochemical examination.

Tumor and Tumor Cell Determination

An immunohistochemical determination of Lewis Y antigen is recommended for the surgically removed tumor tissue. For the histological determination of the Lewis Y antigen, de-paraffinated tissue is incubated with the murine antibody BR55-2 and subsequently with a biotinylated goat anti-mouse IgG (Vector Laboratories). Non-specific binding sites are blocked with normal goat serum. To determine the immune complex, horseradish peroxidase streptavidin (1:325 in phosphate-buffered physiological saline solution) is added, and staining is performed (cf. in this respect J. E. Beesley, chapter 2.5: Avidin-biotin methods in Immunocytochemistry, edited by Oxford Univ. Press, 1993).

Disseminated tumor cells are determined from the blood samples as follows.

1. Tumor Cell Enrichment:

25 ml of peripheral blood were centrifuged in an OncoQuick® tube (Greiner bio-one, Altmünster, Austria) at 1600×g for 20 min at 4° C. The phase containing the tumor cells was transferred into a further centrifuge tube, and a cell pellet was recovered by centrifuging. This pellet was re-suspended. The cellular portion of the suspension was centrifuged onto a slide for a microscopic examination. The slide was stored at −20° C. until evaluation.

2. Tumor Cell Determination

A solution containing fluorescence-labelled specific antibodies was applied to the slide that contained the isolated and enriched tumor cells. After an incubation period of 30 min, the tumor cells labelled by the antibody binding were visualised under the fluorescence microscope (Axioplan Zeiss, Jena, Germany) and counted. The content of tumor cells in the blood was calculated according to the enrichment factor. The method was validated with standard tumor cell suspensions.

As the labelled specific antibody, e.g. IGN311 (Anti-Lewis Y) and A45-B/B3 Anti-Cytokeratin (200 μg/ml, Micromet, Martinsried, Germany), both conjugated with fluorescent proteins, were used.

### Example 2

### Inhibition of the Tumor Growth by Antibodies Against Lewis-Y in Xenograft Models in Naked Mice

The antibody IGN311 (humanized IgG1, according to EP 0 528 767, e.g.) and its murine precursor ABL364 (IgG3, EP 0 547 079), respectively, recognize the Lewis Y antigen, a difucosylated lactosamine-glycoside residue on surface molecules. In some cells, in particular in tumor cells, also receptors for growth factors are modified with Lewis Y antigen. This also applies to the family of the EGF receptors (erbB1, erbB2, erbB3, erbB4, cf. A995/2002). Therefore, it shall be examined with corresponding human tumor cell lines (A431) whether IGN311 also suppresses the establishment of tumors in vivo, in a xenograft model, inhibits the growth of an existing tumor, and prevents a disseminated disease. By comparing the murine precursor ABL364 with IGN311, it shall also be examined whether or not the fixing of complement components plays a role in the elimination of the tumor cells.

It is, i.a., an object to prove experimentally an effectiveness of IGN311 in epithelial tumor models. This provides a prerequisite for the use of IGN311 in the therapy of human diseases.

Material and Methods:

Material:

The fetal calf serum was obtained from PAA Laboratories (Linz, Austria), Dulbecco's modified Eagle Medium (DMEM, RPMI-1640), non-essential amino acids, β-mercaptoethanol, were obtained from GIBCO-BRL (Grand Island, N.Y.). The A431 cell line was purchased from ATCC (Manassas, Va.). L-glutamine, penicillin G and streptomycin were obtained from Sigma Chemical Co.

Cell culture: A431 cells (human epidermal cancer cell line from vulva carcinoma) were cultured in Dulbecco's modified Eagle Medium (DMEM), containing 10% calf serum, 4 mM L-glutamine, 100 units/ml of penicillin G and 100 μg/ml of streptomycin at 5% CO₂ and 37° C. SKBR3 cells (from human breast cancer) were cultured in RPMI-1640 medium at 5% CO₂ and 37° C., wherein the medium was admixed with 10% calf serum, 2 mM L-glutamine, 100 units/ml of penicillin G and 100 μg/ml of streptomycin.

Keeping of animals, xenograft inoculation: pathogen-free female BALB/C nu/nu mice, 5-6 weeks old, were used for the example (source: Versuchstierzucht (test animal husbandry) Himberg). The animals had a weight of from 18 to 20 g.

Because of their immunodeficiency, BALB/c (nu/nu) mice must be kept under pathogen-free conditions in special filter cages (Seal-Safe-IVC cages, Techniplast, Munich). Cages with bedding, drinking water and feed were autoclaved. Manipulations on the test animals were carried out in a sterile workbench. The injected cell lines were checked for contamination with mycoplasmas (a PCR kit being available for this purpose). Culture supernatants of A431 cells were admixed to these murine cell cultures on a test basis and checked for a cytopathic effect; if the latter occurs, it is seen as an indication of the presence of a viral contamination. Naturally, these cells are not administered to the naked mice before the problem of the viral contamination has been eliminated.

Generating Different Tumors in Naked Mice

In 6 mice each per group, ˜5*10⁶ tumor cells are injected subcutaneously (s.c.) into the region of the left flank:

For this purpose, the A431 cells are re-suspended in 200 μl of phosphate-buffered saline solution; after 6 weeks at the latest, the mice are sacrificed by means of cervical dislocation and evaluated.

The administration of the antibodies is by intraperitoneal injection (twice per week). The first administration is on the next day, i.e. approximately 21 hours (24 hours at the most) after application of the tumor cells.

The animals are treated with IGN311 for 4 weeks. Subsequently, the inner organs (in particular the lung) are examined for the formation of metastases. The quantification is by counting the visible lesions.

Data Analysis

The measurement parameter is the tumor size; the latter is determined both via the weight of the tumor (=at the end) and calculated from its dimensions (twice per week); the latter is effected on the basis of the measurement of the (largest) longitudinal and transverse diameter and the thickness, according to the formula for the volume of an ellipsoid (12): volume=length*width*height*π/6, or with smaller tumors whose thickness cannot be determined with certainty: volume=length*width2*π/6. Alternatively, the weight of the tumors is determined.

Results:

At the beginning, a titration study was carried out so as to determine the number of the tumor cells which is necessary for the inoculation. The range was between 0.5-5×10⁶ cells. Tumors were obtained with the A431 cells.

4 mice/group were inoculated with 1×10⁶ A431 cells, the administration of the antibodies was on the next day in an amount of 10 mg/kg of IGN311 or ABL364, respectively.

FIG. 1 shows the inhibition of the growth of the tumor xenograft by IGN311 and ABL364. The administration of the antibodies was started within a day after the subcutaneous tumor cell application.

On day 25 the animals were sacrificed, the tumor mass was removed and its weight determined.

In a further test series, 3×10⁶ tumor cells were inoculated, which led to a rapid tumor formation. Here it was tested whether an increase in the IGN311 dose from 10 mg/kg to 30 mg/kg would lead to an additional growth inhibition. As is apparent from FIG. 2, IGN311 shows an inhibiting activity also at an increased dose.

The determination of the tumor volume was found to be difficult since the tumors did not grow as an ellipsoidal body. Therefore, the animals were sacrificed on day 16, and the weight of the tumor mass was determined. The results can be taken from FIG. 3.

Based on the data obtained, it can be stated that in this xenograft model, IGN311 exhibits a significant anti-tumor activity in vivo and that the effect of the IGN311 is not dramatically increased by increasing its dose from 10 mg/kg to 30 mg/kg.

In an alternative test set-up, the proof of the inhibition of tumor growth and of the dissemination of the tumor cells, respectively, by the antibodies is provided in that the antibodies are administered within 24 hours at the most before the tumor cells are applied subcutaneously or intravenously. Again, the tumor cells may be applied subcutaneously, intravenously or directly into the lung of the test animals.

On the live animals, the tumor growth is determined via the measurement of the tumor size, after several days or weeks the animals are sacrificed by cervical dislocation, and the tumor size is determined. Furthermore, the organs are removed and examined for micro-metastases and other tumor-caused changes by means of various methods.

### Example 3

### Proof of the Reduction of Circulating EpCAM Cells in Blood

Patients, Material and Methods:

A murine IgG2a Mab having EpCAM mimick properties was used for the vaccination. The antibody was subcutaneously administered in an amount of 0.5 mg Mab, adsorbed on 1.67 mg of aluminum hydroxide in 0.5 ml of buffer.

As test subjects, patients suffering from cancer (more than 19 years of age) were chosen in whom conventional therapy was unsuccessful. The treatment was effected with 0.5 mg of antibody subcutaneously on days 1, 15, 29, 57.

Analysis of Circulating Tumor Cells in Peripheral Blood:

5 ml of heparinized blood were drawn on days 1, 29, and 71. After a mild erythrocyte lysis, the residual fluid was incubated with para-magnetic anti-EpCAM antibody, and the cells were separated on a magnetic column (Miltenyi). After the elution, the bound cells were labelled with FITC anti-EpCAM antibody and analysed, or counted in an inversion fluorescence microscope, respectively.

The results can be taken from FIG. 4 in which the reduction of the circulating EpCAM cells in blood is clearly visible.

### Example 4

### Administration of IGN311 to Patients with Pleural Effusion

In a clinical study, 100 mg of IGN311 is administered intravenously in a single dose to patients (from 18 to 80 years of age) who suffer from cancer and have a pleural effusion, 24 hours at the most before removal of the effusion.

Both the volume of the pleural fluid which is removed by draining and the presence of Lewis y positive tumor cells are determined. The biological activity of the IGN311 in the pleural effusion can be analyzed by CDC determination. Moreover, it can be determined whether or not the time until the pleurodesis can be carried out, i.e. until the pleural fluid has been drained, can be shortened by administering the antibody.

1. A method for the intra-operative treatment of a tumor to inhibit dissemination of tumor cells, which comprises administering to the patient an antibody directed against a tumor-associated antigen during an intra-operative treatment whereby immunocomplexing of tumor cells within the scope of the surgical intervention inhibits dissemination of tumor cells.

2. The method according to claim 1, wherein the antibody is directed against an epitope of a surface antigen of a tumor cell.

3. The method according to claim 1 or 2, wherein the tumor cell is an epithelial tumor cell.

4. The method according to claim 1, wherein the antibody is directed against an epitope of an antigen selected from the group consisting of peptides, proteins, carbohydrates, and glycolipids.

5. The method according to claim 1, wherein the antibody is in an antibody mixture of various antibodies having a specificity for tumor-associated antigens.

6. The method according to claim 1, wherein the antibody functionally activates the immune system, according to an ADCC and CDC effector function.

7. The method according to claim 1, wherein the antibody binds to the tumor-associated antigen with an affinity corresponding to a dissociation constant below a Kd value of 10⁻⁶ mol/l.

8. The method according to claim 1, characterised in that the antibody is derived from murine, chimeric, humanized and/or human sources.

9. The method according to claim 1, wherein the medicament is systemically used with a single dose of at least 50 mg per patient.

10. The method according to claim 1, wherein the medicament is locally applied to the tumor tissue and/or to the wound area.

11. The method according to claim 1, wherein the medicament is administered immediately during or before the surgical intervention.

12. The method according to claim 1, wherein the surgical intervention is carried out for a biopsy and/or for the removal of a solid tumor.

13. The method according to claim 1, wherein the surgical intervention is carried out for a determination regarding the malignancy of a tumor.

14. The method according to claim 1, wherein the antibody is determined on the immunocomplexed tumor tissue after the surgical intervention.

15. The method according to claim 1, wherein the antibody is determined on tumor cells in blood or serum samples.

16. A kit for the intra-operative treatment of tumor patients, comprising
a) a medicament based on an antibody directed against a tumor-associated antigen, and
b) a means for the diagnostic determination of malignant tumor cells which are immunocomplexed with the antibody.

17. The method according to claim 4, wherein the antigen is a member selected from the group consisting of EpCAM, NCAM, CEA, Lews Y, Sialyl-TN, Globo H, GD2, GD3 and GM2.

18. The method according to claim 7, wherein said Kd value is 10⁻⁷ mol/l.

19. The method according to claim 7, wherein said Kd value is 10⁻⁸ mol/l.

20. The method according to claim 8, wherein said single does is at least 100 mg.

21. The method according to claim 8, wherein said single does is at least 200 mg.

22. The method according to claim 8, wherein said single does is at most 2 mg.

23. The method according to claim 11, wherein the medicament is administered within 24 hours before the surgical intervention.

24. The method according to claim 11, wherein the medicament is administered within 4 hours before the surgical intervention.
